(19) United States
(12) Patent Application Publication
(10) Pub. No.: US 2024/0312585 A1
(43) Pub. Date: Sep. 19, 2024

(54) AUTOMATIC TASK OPTIMIZATION METHODS IN PRODUCTION AND RESEARCH FACILITIES

(71) Applicant: Yokogawa Electric Corporation, Tokyo (JP)

(72) Inventors: Yasuo ASAMI, Bedok (SG); Venkata krishnaprasad Reddy VURUBINDI, Bedok (SG); Harish Reddy ARUMALLA, Bedok (SG); Manoj SHIVASHANKAR, Bedok (SG)

(21) Appl. No.: 18/121,179

(22) Filed: Mar. 14, 2023

(51) Int. Cl. G16H 20/10

(52) U.S. Cl. CPC G16H 20/10

(57) ABSTRACT

Electronic Laboratory notebooks allow a process, such as an experiment, to be defined and observations during and/or after the experiment to be recorded. The steps to perform an experiment may be saved as a template to enable repeating the same process or, with modification, easily allow a new template to be created without requiring rewriting the experiment's workflow from scratch. This is a method for improving a work process by optimizing its workflow. The method involves identifying multiple work steps associated with the workflow, creating a first template that includes a subset of these work steps, and duplicating the first template to make a first duplicated template. The method also uses artificial intelligence to further optimize the resources.

### FIELD OF THE DISCLOSURE

The invention relates generally to systems and methods for an electronic laboratory notebook and particularly to optimizing workflows comprising a number of steps.

### BACKGROUND

Paper-based lab notebooks and electronic laboratory or “lab” notebooks are commonly used to plan laboratory projects, how they were carried out, and observations. An accurate lab notebook provides a record to enable others to reproduce the experiment or other laboratory project and obtain the same result. If the same result is not obtained, then the equipment utilized, reagents and quantities used, environmental conditions, etc. can be investigated. In addition to the reaction itself, lab notebooks often contain records of those involved and the particular operations each person performed.

Lab notebooks can play an important role in determining the commercial feasibility of a process and who is an inventor of a new substance, as well as a record of processes that failed to produce a desired result or produced an unexpected beneficial result.

Lab notebooks are often only a part of the laboratory's operations. Equipment and personnel may need to be scheduled as well as the workflow itself. For example, an experiment may involve repeating an operation many times, but with a variations, such as the concentration of a solution being incremented each time. A result from the experiments may be instantly available or not be available for some time. If an experiment requires the use of a limited resource (e.g., personnel or a piece of equipment that is in high demand), it may not be possible to perform one experiment, wait until a result is available, and then perform a subsequent experiment based on the result. Having a workflow that both considers the experiments themselves and the other inputs (e.g., equipment, personnel, reagents, energy, etc.) is important to efficient operations of many labs.

### SUMMARY

The prior art of laboratory notebooks are invaluable tools. Despite the benefits, connecting production to research has been absent.

These and other needs are addressed by the various embodiments and configurations of the present invention. The present invention can provide a number of advantages depending on the particular configuration. These and other advantages will be apparent from the disclosure of the invention(s) contained herein.

In one embodiment, systems and methods are provided to manage projects, documents, assets (e.g., users, equipment, etc.), permissions, experiments, and scheduling of equipment and personnel is provided. Additionally or alternatively, laboratory operations may be maintained as a template to reproduce an experiment identically or with a desired variation. Data produced by an experiment may be analyzed in real-time or stored in a data repository (e.g., server, cloud, server farm, data lake, etc.) for subsequent analysis.

In another embodiment, data is sorted in accordance with template. Sorting of pre-registered template data is performed for users according to various projects, and the most suitable and essential tasks are automatically displayed on the dashboard or in each task list. The most suitable and essential tasks are automatically prioritized for display on the dashboard or in each task list.

In another embodiment, storage of data in data repository is provided. A data repository, such as a data lake, is used to store documents, numerical and image data from all departments including production and research with a unique ID, and automatically consolidate them into a project management list or dashboard.

In another embodiment, branching-out of templates to suit other workflow steps is provided. Each step of the workflow is stored in the data lake as a template. Each stored step can be freely combined using building blocks to create a new template easily according to the workflow.

In another embodiment, results from the execution of tasks are saved as a reusable template. When used as a template, measurement conditions, operating procedures, etc. can be modified manually or automatically using AI, and used to create optimal business processes and experimental methods. This enables optimization and efficiency improvement of business processes in a simple step.

After using a template as a task, a template can be upgraded and saved as modified template. This allows the above procedure to be repeated. This enables process optimization by repeating the above steps.

Various data from different departments can be stored and integrated in a data lake to share documents and data with different departments.

When conducting experiments in the lab, experimental methods are saved as templates. Frequently used templates or tasks are automatically prioritized on the dashboard, and these templates are versioned and optimized for evaluation or more efficient methods.

The research department studies the best analysis and evaluation methods and stores them as standard operating procedure (SOP) templates. This analysis and evaluation method is then used as an SOP for work performed in the quality control department.

When process evaluation is performed in the manufacturing department, the procedures saved as templates are executed, the workflow is optimized, and the results are saved as SOP templates. This SOP template can also be used in the manufacturing department as a procedure manual for production.

Systems and methods are provided to unify a plurality of electronic and paper-based systems to provide a single point of information and management. Paper records are digitized maintained electronically. Digitation may comprise imaging and/or content extraction (e.g., optical character recognition) which may further provide searchable content.

In another embodiment, workflows are determined to comply with legal requirements (e.g., FDA, CFR Part 11, etc.), resource availability, etc. Workflows can be maintained as a modifiable template to alleviate one or more steps required to develop subsequent workflows. In one embodiment, a system is disclosed, comprising:

A method of optimizing a workflow relating to a work process, the method comprising: identifying a plurality of work steps associated with the workflow; creating a first template, wherein the first template comprises a first subset of the plurality of work steps; duplicating the first template to create a first duplicated template; modifying at least one of the first subset of the plurality of work steps automatically in the first duplicated template in accordance with a proposed modification workflow while maintaining a second subset of the plurality of work steps that remained unchanged; automatically routing the proposed modification workflow through to the first duplicated template while allowing the workflow to be automatically routed to the first template; and validating the proposed modification workflow using the at least one of the first subset of the plurality of work steps and the second subset of the plurality of work steps that remained unchanged.

A method of executing a workflow, comprising: accessing a first workflow comprising a number of steps wherein each step of the number of steps comprises an assigned resource and a time for performance thereof; creating a second workflow comprising a modification to the first workflow comprising at least a modification to the number of steps comprising adding a new step, a deleted step of the number of steps, or a modification to a step of the number of steps and wherein the modification to the first workflow reduces a workflow resource; executing an executable workflow comprising the second workflow upon determining that, for the second workflow, each of the number of steps comprises the assigned resource that is indicated by a data record in a database as being available during the time; and executing the executable workflow comprising the first workflow upon determining that, for the second workflow, each of the number of steps comprises the assigned resource that is indicated by the data record in the database as not available during the time.

A system, comprising: at least one processor coupled to computer memory comprising computer-executable instructions; and wherein the at least one processor performs: accessing a first workflow comprising a number of steps wherein each step of the number of steps comprises an assigned resource and a time for performance thereof; creating a second workflow comprising a modification to the first workflow comprising at least a modification to the number of steps comprising adding a new step, a deleted step of the number of steps, or a modification to a step of the number of steps and wherein the modification to the first workflow reduces a workflow resource; executing an executable workflow comprising the second workflow upon determining that, for the second workflow, each of the number of steps comprises the assigned resource that is indicated by a data record in a database as being available during the time; and executing the executable workflow comprising the first workflow upon determining that, for the second workflow, each of the number of steps comprises the assigned resource that is indicated by the data record in the database as being not available during the time.

A method of optimizing a workflow relating to a work process, the method comprising: identifying a plurality of work steps associated with the workflow; creating a first template, wherein the first template comprises a first subset of the plurality of work steps; duplicating the first template to create a first duplicated template; modifying at least one of the first subset of the plurality of work steps automatically in the first duplicated template in accordance with a proposed modification workflow while maintaining a second subset of the plurality of work steps that remained unchanged; automatically routing the proposed modification workflow through to the first duplicated template while allowing the workflow to be automatically routed to the first template; validating the proposed modification workflow using the at least one of the first subset of the plurality of work steps and the second subset of the plurality of work steps that remained unchanged; and replacing the first template with the first duplicated template when a result of validating the proposed modification workflow exceeds a predetermined threshold.

A method, comprising: accessing a first workflow comprising a number of steps wherein each step of the number of steps comprises an assigned resource and a time for performance thereof; generating a workflow template from the first workflow; receiving experimental inputs; generating, based on the experimental inputs, a second workflow that is different from the first workflow, wherein the second workflow comprises at least one step that is different from the number of steps in the first workflow; comparing a performance of the workflow template with a performance of the second workflow; and updating a workflow template database based on the comparison of the performance of the workflow template with the performance of the second workflow.

Exemplary aspects are further directed to:

Any of the foregoing comprising replacing the first template with the first duplicated template.

Any of the forgoing comprising creating a second template, wherein the second template comprises a second subset of the plurality of work steps; duplicating the second template to create a second duplicated template; modifying at least one of the second subset of the plurality of work steps automatically in the second duplicated template in accordance with a proposed modification workflow while maintaining a second subset of the plurality of work steps that remained unchanged; automatically routing the proposed modification workflow through to the second duplicated template while allowing the workflow to be automatically routed to the second template; and validating the proposed modification workflow using the at least one of the second subset of the plurality of work steps and the second subset of the plurality of work steps that remained unchanged.

Any of the foregoing wherein a subset of a pharmaceutical screening workflow is formed, the pharmaceutical workflow comprises: a research work step of creating a proposed design schedule and a proposed formulation based on the first duplicated template; and an analysis work step of validating the proposed design schedule and the proposed formulation based on an automatically generated analysis template that is generated from the first duplicated template.

Any of the foregoing wherein the pharmaceutical screening workflow further comprising: an in-vivo work step of preparing cells for culture process.

Any of the foregoing wherein the pharmaceutical screening workflow further comprising: a synthesis work step of preparation and synthesis of drug administration based on an automatically generated synthesis template generated from the first duplicated template.

Any of the foregoing wherein the in-vivo work step of preparing cells further comprises a first sub-step of receiving a synthesized drug sample from the synthesis work step, and a second sub-step of preparing dosage and cell culture based on an information of the automatically generated synthesis template.

Any of the foregoing wherein the in-vivo work step of preparing cells further comprises a third sub-step of collecting sample information of the cultured cells into a modified first duplicated template.

Any of the foregoing wherein validating the proposed modification workflow comprises fourth sub-step of analysis of a sample batch of cells from culture process.

Any of the foregoing wherein the proposed design schedule and the proposed formulation based on the first duplicated template are generated using a trained neural network.

Any of the foregoing wherein the trained neural network is used to perform a fifth sub-step of review of the sample batch of cells from culture process and in accordance with the proposed design schedule and the proposed formulation based on the first duplicated template.

Any of the foregoing further comprising replacing the first template with a hybrid template comprising at least one first selected work step from the first subset and at least one second selected work step from the second subset.

Any of the foregoing comprising wherein the workflow is a pharmaceutical workflow.

Any of the foregoing comprising wherein the creating the second workflow comprises providing the number of steps to a neural network trained to minimize utilization of a workflow resources for the first workflow when provided with the number of steps and, in response, returning the modification.

Any of the foregoing comprising wherein the neural network is trained to determine a reduced resource utilization for a target workflow when provided with steps of the workflow, comprising:

Any of the foregoing comprising accessing a set of past workflows, each comprising a plurality of past steps, and each past step having a corresponding past resource; applying one or more transformations to each of the set of past workflows, including one or more of adding a step, removing a step, altering a past resource to utilize an equivalent resource able to perform the past step, utilizing more of a different resource to perform a step, utilizing a different input to perform a step, combining two or more past steps utilized by different resources into a single step utilized by a common resource, or utilizing less of the different resource to create a modified set of past steps; creating a first training set comprising the set of past steps, the modified set of past steps, and a set of steps able to perform the target workflow that does not reduce resource utilization for the execution thereof; training the neural network in a first training stage using the first training set; creating a second training set for a second state of training comprising the first training set and the set of steps able to perform the target workflow that does not reduce resource utilization that was incorrectly identified as reducing resource utilization in the first training stage; and training the neural network in the second state using the second training set.

Any of the foregoing comprising wherein workflow resources of the workflow comprise at least one of a machine, a consumable input, time, a waste product, power, or variation of an output product.

Any of the foregoing comprising wherein executing the executable workflow comprises allocating the assigned resource at the respective corresponding time.

Any of the foregoing comprising wherein executing the executable workflow comprises executing at least one assigned resource at the corresponding time wherein the assigned resource comprises a machine.

Any of the foregoing comprising wherein the time for one of the number of steps comprises a relative time determined by the completion of a prerequisite step of the number of steps.

Any of the foregoing comprising further comprising saving the executable workflow as a template.

Any of the foregoing comprising wherein at least one of an input product utilized by the template or output product produced by the template is modified by the template.

Any of the foregoing comprising further comprising, upon at least one of starting, partially completing, or completing execution of at least one of the number of steps, writing indicia thereof as a status of the at least one of the number of steps to a record of data storage.

Any of the foregoing comprising wherein a workflow status is presented to a user device comprising displaying a status corresponding to the indicia.

Any of the foregoing comprising wherein the workflow resource comprises at least one of a machine, a consumable input, time, a waste product, power, or variation of an output product.

Any of the foregoing comprising wherein the at least one processor performs executing the executable workflow further comprising allocating the assigned resource at the respective corresponding time.

Any of the foregoing comprising wherein the at least one processor performs creating the second workflow further comprising providing the number of steps to a neural network trained to minimize utilization of a workflow resources for the first workflow when provided with the number of steps and, in response, returning the modification.

Any of the foregoing forming a subset of a pharmaceutical screening workflow, the pharmaceutical workflow comprises: a research work step of creating a proposed design schedule and a proposed formulation based on the first duplicated template; and an analysis work step of validating the proposed design schedule and the proposed formulation based on an automatically generated analysis template that is generated from the first duplicated template.

Any of the foregoing wherein the pharmaceutical screening workflow further comprising: an in-vivo work step of preparing cells for culture process.

Any of the foregoing wherein the pharmaceutical screening workflow further comprising: a synthesis work step of preparation and synthesis of drug administration based on an automatically generated synthesis template generated from the first duplicated template.

Any of the foregoing wherein the in-vivo work step of preparing cells further comprises a first sub-step of receiving a synthesized drug sample from the synthesis work step, and a second sub-step of preparing dosage and cell culture based on an information of the automatically generated synthesis template.

Any of the foregoing wherein the in-vivo work step of preparing cells further comprises a third sub-step of collecting sample information of the cultured cells into a modified first duplicated template.

Any of the foregoing wherein validating the proposed modification workflow comprises fourth sub-step of analysis of a sample batch of cells from culture process.

Any of the foregoing wherein the proposed design schedule and the proposed formulation based on the first duplicated template are generated using a trained neural network.

Any of the foregoing wherein the trained neural network is used to perform a fifth sub-step of review of the sample batch of cells from culture process and in accordance with the proposed design schedule and the proposed formulation based on the first duplicated template.

wherein the performance of the workflow template comprises at least one of an efficiency and an efficacy associated with completing a task using the workflow template and wherein the performance of the second workflow comprises at least one of an efficiency and an efficacy associated with completing the task using the second workflow.

Any of the foregoing wherein the performance of the second workflow is better than the performance of the workflow template, the method further comprising: providing a display via a display device that the second workflow is prioritized relative to the workflow template.

Any of the foregoing wherein the performance of the second workflow is better than the performance of the workflow template, the method further comprising: generating a second workflow template from the second workflow; and replacing the workflow template with the second workflow template.

Any of the foregoing wherein the performance of the second workflow is better than the performance of the workflow template, the method further comprising: generating a second workflow template from the second workflow; and presenting a prioritized list of workflow templates, wherein the prioritized list indicates that the second workflow template comprises a higher priority as compared to the workflow template.

Any of the foregoing wherein the comparing comprises performing a task with the workflow template and with the second workflow in parallel, then comparing an outcome of the task based on performing the task with the workflow template against an outcome of the task based on performing the task with the second workflow.

Any of the foregoing wherein the comparing is performed with an artificial intelligence engine.

Any of the foregoing further comprising automatically saving a second workflow template generated from the second workflow in response to the artificial intelligence engine determining that the outcome of the task is better when performing the task with the second workflow and in response to the artificial intelligence engine providing a confidence score that exceeds a predetermined threshold.

Any of the foregoing further comprising modifying the workflow template with at least one step from the second workflow.

Any of the foregoing wherein modifying the workflow template comprises updating at least one step of the workflow template with a step from the second workflow.

Any of the foregoing further comprising presenting a workflow status via a user device, wherein the workflow status provides an indication of a performance of the workflow template relative to a performance of the second workflow.

A system on a chip (SoC) including any one or more of the above of the embodiments described herein.

One or more means for performing any one or more of the above aspects of the embodiments described herein.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

Any of the above aspects, wherein the data storage comprises a non-transitory storage device, which may further comprise at least one of: an on-chip memory within the processor, a register of the processor, an on-board memory co-located on a processing board with the processor, a memory accessible to the processor via a bus, a magnetic media, an optical media, a solid-state media, an input-output buffer, a memory of an input-output component in communication with the processor, a network communication buffer, and a networked component in communication with the processor via a network interface.

It is to be appreciated that any feature described herein can be claimed in combination with any other feature(s) as described herein, regardless of whether the features come from the same described embodiment.

The phrases “at least one,” “one or more,” “or,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B, and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible, non-transitory medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

The term “means” as used herein shall be given its broadest possible interpretation in accordance with 35 U.S.C., Section 112(f) and/or Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The preceding is a simplified summary of the invention to provide an understanding of some aspects of the invention. This summary is neither an extensive nor exhaustive overview of the invention and its various embodiments. It is intended neither to identify key or critical elements of the invention nor to delineate the scope of the invention but to present selected concepts of the invention in a simplified form as an introduction to the more detailed description presented below. As will be appreciated, other embodiments of the invention are possible utilizing, alone or in combination, one or more of the features set forth above or described in detail below. Also, while the disclosure is presented in terms of exemplary embodiments, it should be appreciated that an individual aspect of the disclosure can be separately claimed.

### BRIEF DESCRIPTION OF THE DRAWINGS

The present disclosure is described in conjunction with the appended figures:

FIG. 1 depicts a system in accordance with embodiments of the present disclosure;

FIG. 2 depicts a process in accordance with embodiments of the present disclosure;

FIG. 3 depicts a process in accordance with embodiments of the present disclosure;

FIG. 4 depicts a system in accordance with embodiments of the present disclosure;

FIG. 5 depicts a process in accordance with embodiments of the present disclosure; and

FIG. 6 depicts a process in accordance with embodiments of the present disclosure.

### DETAILED DESCRIPTION

The ensuing description provides embodiments only and is not intended to limit the scope, applicability, or configuration of the claims. Rather, the ensuing description will provide those skilled in the art with an enabling description for implementing the embodiments. It will be understood that various changes may be made in the function and arrangement of elements without departing from the spirit and scope of the appended claims.

Any reference in the description comprising a numeric reference number, without an alphabetic sub-reference identifier when a sub-reference identifier exists in the figures, when used in the plural, is a reference to any two or more elements with the like reference number. When such a reference is made in the singular form, but without identification of the sub-reference identifier, it is a reference to one of the like numbered elements, but without limitation as to the particular one of the elements being referenced. Any explicit usage herein to the contrary or providing further qualification or identification shall take precedence.

The exemplary systems and methods of this disclosure will also be described in relation to analysis software, modules, and associated analysis hardware. However, to avoid unnecessarily obscuring the present disclosure, the following description omits well-known structures, components, and devices, which may be omitted from or shown in a simplified form in the figures or otherwise summarized.

For purposes of explanation, numerous details are set forth in order to provide a thorough understanding of the present disclosure. It should be appreciated, however, that the present disclosure may be practiced in a variety of ways beyond the specific details set forth herein.

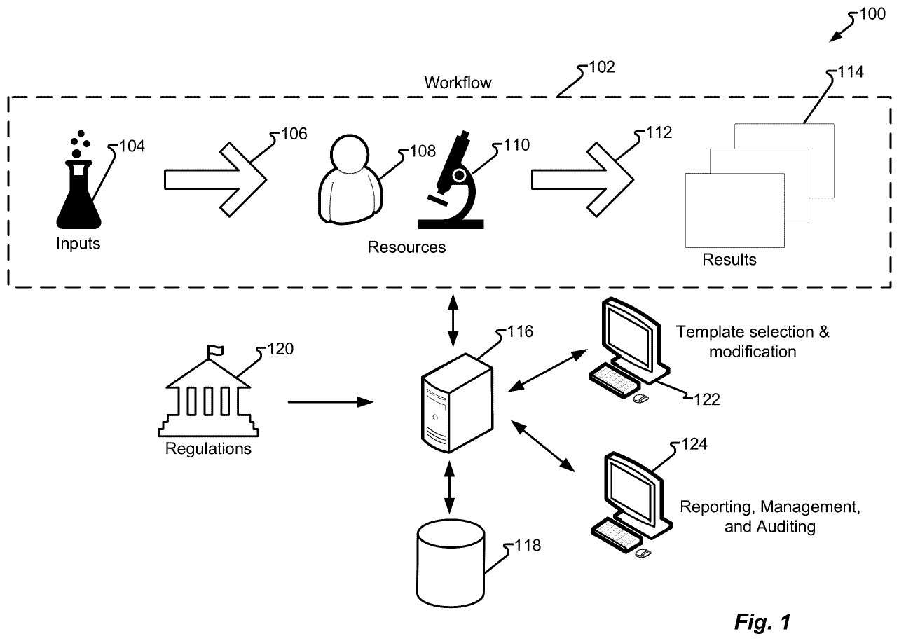

FIG. 1 depicts system 100 in accordance with embodiments of the present disclosure. System 100 manages one or more templates of workflow 102, such as an experiment similar study. In order to perform an experiment to produce a result, workflow 102 comprises a number of templates. Workflow 102 may comprise a number of work steps (e.g., gathering inputs, setting up equipment, performing the experiment, making observations, etc.) any one or more may be maintained as a template for future use or modification. Templates may be utilized for one or more, and preferably all, steps of an experiment. For example, a template may acquire inputs 104 (e.g., consumable supplies, reagents, etc.) for provisioning 106 to human resource 108 and/or equipment resource 110. Once all inputs 104 and resources (e.g., human resource 108, equipment resource 110) are available (or will be available when needed) a template may be utilized in experiment performance 112 and thereby producing results 114.

In another embodiment, templates are created and modified by server 116 which maintains the templates in data storage 118. Data storage 118 may comprise a single data storage device, storage array, cloud, data lake, etc. Similarly, server 116 may comprise a single device (e.g., personal computer), server, server farm, server array, cloud, etc. Server 116 may access or be provided with rules, such as rules from regulatory requirements 120. Regulatory requirements 120 may be governmental requirements, industry requirements, best practices, customer requirements, and/or other source of controlling information outside of the processes itself. For example, an experiment may require the combining of two substances and analyzing the end product. However, regulatory requirements 120 may require additional or alternative steps, such as analyzing the end product utilizing a particular method or performing additional steps, such as reporting temperatures and pressures at specified intervals.

For clarity, template selection and modification 122 and reporting, management, and auditing 124 are illustrated as discrete workstations interfacing with server 116, such as via a network (not shown). In other embodiments, two or more of server 116, template selection and modification 122, and reporting, management, and auditing 124 workstations may be combined into a single device or divided into more devices.

In one embodiment, no templates for workflow 102 exist. Accordingly, an operator of template selection and modification 122 may create a new template for storage on data storage 118. The operator of template selection and modification 122 is variously embodied and may include a human operator, an artificial intelligent (AI) operator, or a combination thereof, such as when a human operator selects a particular experiment and an AI operator selects known inputs 104 and processes, apply regulatory steps from regulatory requirements 120, and/or other operations. The human operator may the modify the template provided by the AI operator and vice versa.

As a benefit, workflow 102 may be verified (e.g., all required inputs 104, human resource 108, server 116, etc. are or will be available for experiment performance 112). Accordingly, server 116 may access one or more other systems (e.g., resource scheduling, inputs 104 ordering/scheduling, etc.) if not otherwise available, such as via common storage on data storage 118. If, for example, a particular piece of equipment (e.g., one of equipment resource 110) when needed, one or more templates of workflow 102 may be altered so that other requirements (e.g., inputs 104, human resource 108) are made available for other purposes until such time as equipment resource 110 is available. As a benefit, experiments may be coordinated to avoid be made available only to sit idle and/or double booking.

In another embodiment, reporting, management, and auditing 124 may receive end results (e.g., results 114) or one or more intermediate results produced by experiment performance 112. The new or modified templates may then be saved in data storage 118.

In another embodiment, an experiment may access data storage 118 to retrieve an existing template. The template may be modified by template selection and modification 122 (human and/or AI). For example, a stored template may perform a reaction to completion in five minutes. A human operator may attempt to reduce the reaction time by selecting a different input 104 or steps of a process. The original operation may require heat from a particular equipment resource 110 but the modified experiment requires a greater heat and, therefore, a different equipment resource 110. The resulting template(s) is saved and may be made available as workflow 102.

In another embodiment reporting, management, and auditing 124 may have access to reports, logs, or other information such as specific readings, lot numbers of inputs 104, identity of human resource 108, settings of equipment resource 110, etc.

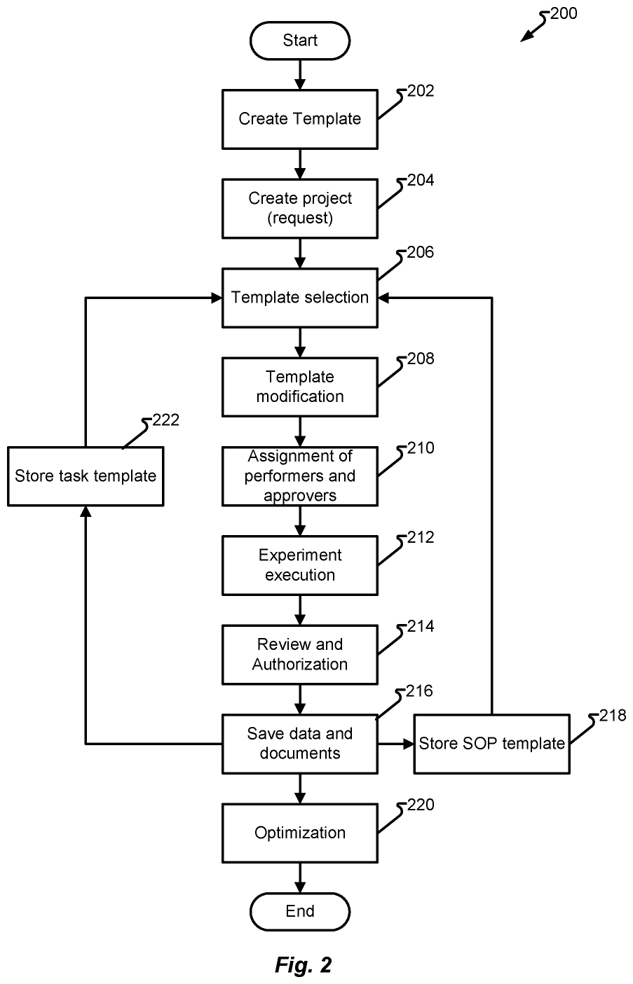

FIG. 2 depicts process 200 in accordance with embodiments of the present disclosure. In one embodiment, process 200 is embodied as machine-readable instructions maintained in a non-transitory memory that when read by a machine, such a processors of a server, cause the machine to execute the instructions and thereby execute process 200. The processor of the server may include, but is not limited to, at least one processor of server 116.

Process 200 begins and step 202 creates a template. The template prescribes one or more of a step, input, process, resource, etc., such as inputs 104, provisioning 106, human resource 108, equipment resource 110, experiment performance 112, or a configuration thereof in order to perform workflow 102.

Step 204 creates a project, such as inputs 104 or other ordered set of templates to produce a result. Step 206 selects a template. The template may be generic, targeted to a specific portion of workflow 102, define the entirety of workflow 102, or any level in between. Step 208 modifies the template, if necessary, to customize workflow 102 for a particular experiment or other limitation thereof (e.g., a particular human resource 108 is not available, the desired equipment resource 110 has limitations that affect inputs 104, etc.).

In another embodiment, step 208 comprises duplicating a template to create a first duplicate template. At least one of the work steps of the first duplicate template are modified. Modification is variously embodied and may include one or more of adding a new step, deleting a step, or modifying a step, or performing a step with a different resource (e.g., inputs 104, equipment resource 110, etc.).

Step 210 obtains the necessary approvals and inputs (e.g., inputs 104) and step 212 performs the experiment (e.g., experiment performance 112). A result is produced which is reviewed and authorized in step 214 and data and documentation saved in step 216.

Optionally, step 218 may save the steps performed in step 212 as a standard operating procedure (SOP) template to be available for selection in another iteration of step 206.

Additionally or alternatively, the template may be stored, such as in data storage 118 and be available for selection in another iteration of step 206. Step 220 performs any optimization necessary.

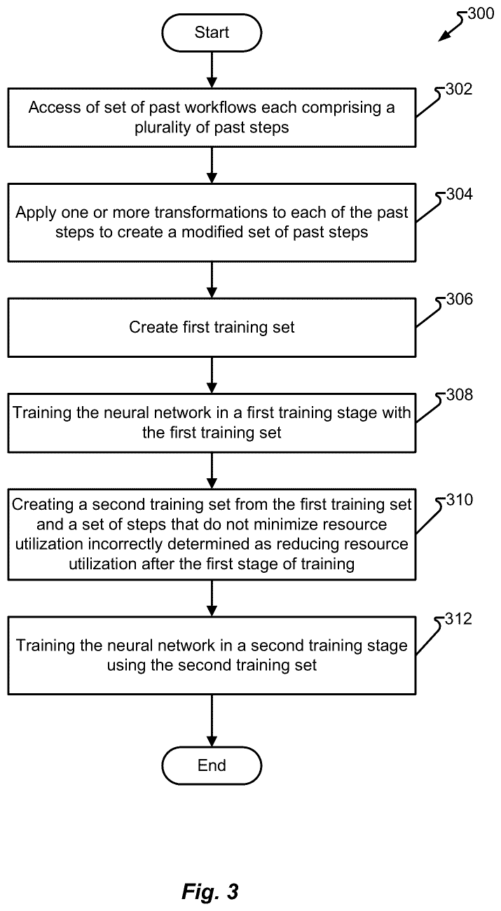

FIG. 3 depicts process 300 in in accordance with embodiments of the present disclosure. In one embodiment, an artificial intelligence (AI), such as a neural network, is utilized to minimize utilization of workflow resources or a portion of a workflow resources, such as the resources defined in a template. Accordingly, a workflow is provided to a trained neural network and an optimized, such as a reduced time, reduced number of steps, reduced usage of a particular input, reduced usage of a particular equipment resource, reduce use of a particular huma resource, reduced energy input, more reliable result, more consistent result, reduced undesirable byproducts, or combinations thereof is obtained.

A neural network, as is known in the art and in one embodiment, self-configures layers of logical nodes having an input and an output. If an output is below a self-determined threshold level, the output is omitted (i.e., the inputs are within the inactive response portion of a scale and provide no output). If the self-determined threshold level is above the threshold, an output is provided (i.e., the inputs are within the active response portion of a scale and provide an output). The particular placement of the active and inactive delineation is provided as a training step or steps. Multiple inputs into a node produce a multi-dimensional plane (e.g., hyperplane) to delineate a combination of inputs that are active or inactive.

Process 300 trains a neural network. Process 300 begins and, in step 302, accesses a past workflows comprising a plurality of past steps. Herein, step may be a process step alone (e.g., heat an input to 250° C.) or a process step for a particular input (e.g., heat input “A” to 250° C.) or a particular result (e.g., mix the combination of input “A” and solution “B” until input “A” is dissolved). Step 304 applies one or more transformation to each of the past steps to create a modified set of past workflows. The modifications comprising one or more of adding a step, removing a step, altering a past resource to utilize an equivalent resource able to perform the past step, utilizing more of a different resource to perform a step, utilizing a different input to perform a step, combining two or more past steps utilized by different resources into a single step utilized by a common resource, or utilizing less of the different resource.

Step 306 creates a first training set comprising the set of past steps, the modified set of pasts steps and a set of steps able to perform the target workflow that does not reduce resource utilization for the execution thereof. Step 308 then trains the neural network with the first training set.

Step 310 creates a second training set for a second state of training comprising the first training set and the set of steps able to perform the target workflow that does not reduce resource utilization that was incorrectly identified as reducing resource utilization in the first training stage. Step 312 then trains the neural network in the second state using the second training set.

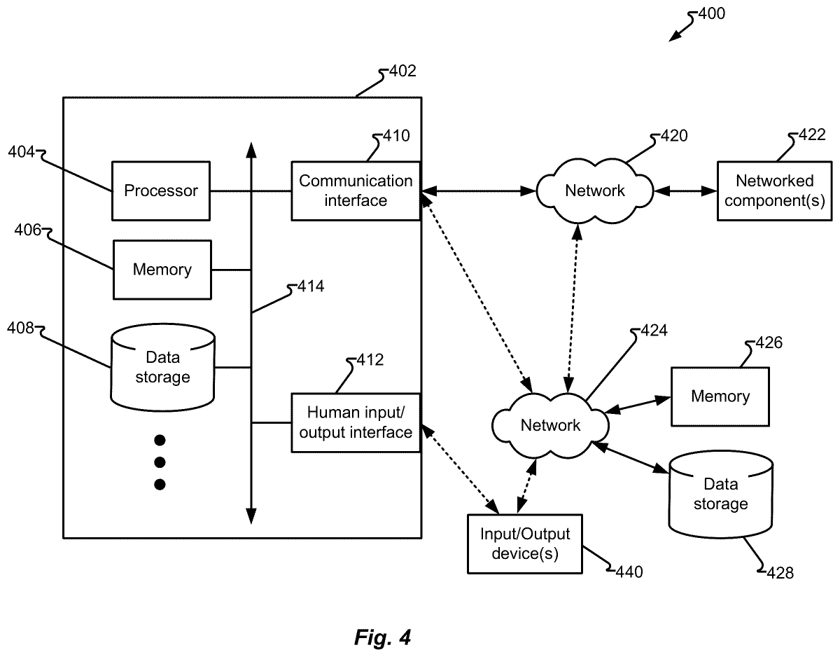

FIG. 4 depicts device 402 in system 400 in accordance with embodiments of the present disclosure. In one embodiment, server 116 may be embodied, in whole or in part, as device 402 comprising various components and connections to other components and/or systems. The components are variously embodied and may comprise processor 404. The term “processor,” as used herein, refers exclusively to electronic hardware components comprising electrical circuitry with connections (e.g., pin-outs) to convey encoded electrical signals to and from the electrical circuitry. Processor 404 may comprise programmable logic functionality, such as determined, at least in part, from accessing machine-readable instructions maintained in a non-transitory data storage, which may be embodied as circuitry, on-chip read-only memory, computer memory 406, data storage 408, etc., that cause the processor 404 to perform the steps of the instructions. Processor 404 may be further embodied as a single electronic microprocessor or multiprocessor device (e.g., multicore) having electrical circuitry therein which may further comprise a control unit(s), input/output unit(s), arithmetic logic unit(s), register(s), primary memory, and/or other components that access information (e.g., data, instructions, etc.), such as received via bus 414, executes instructions, and outputs data, again such as via bus 414. In other embodiments, processor 404 may comprise a shared processing device that may be utilized by other processes and/or process owners, such as in a processing array within a system (e.g., blade, multi-processor board, etc.) or distributed processing system (e.g., “cloud”, farm, etc.). It should be appreciated that processor 404 is a non-transitory computing device (e.g., electronic machine comprising circuitry and connections to communicate with other components and devices). Processor 404 may operate a virtual processor, such as to process machine instructions not native to the processor (e.g., translate the VAX operating system and VAX machine instruction code set into Intel® 9xx chipset code to enable VAX-specific applications to execute on a virtual VAX processor). However, as those of ordinary skill understand, such virtual processors are applications executed by hardware, more specifically, the underlying electrical circuitry and other hardware of the processor (e.g., processor 404). Processor 404 may be executed by virtual processors, such as when applications (i.e., Pod) are orchestrated by Kubernetes. Virtual processors enable an application to be presented with what appears to be a static and/or dedicated processor executing the instructions of the application, while underlying non-virtual processor(s) are executing the instructions and may be dynamic and/or split among a number of processors.

In addition to the components of processor 404, device 402 may utilize computer memory 406 and/or data storage 408 for the storage of accessible data, such as instructions, values, etc. Communication interface 410 facilitates communication with components, such as processor 404 via bus 414 with components not accessible via bus 414. Communication interface 410 may be embodied as a network port, card, cable, or other configured hardware device. Additionally or alternatively, human input/output interface 412 connects to one or more interface components to receive and/or present information (e.g., instructions, data, values, etc.) to and/or from a human and/or electronic device. Examples of input/output devices 430 that may be connected to input/output interface include, but are not limited to, keyboard, mouse, trackball, printers, displays, sensor, switch, relay, speaker, microphone, still and/or video camera, etc. In another embodiment, communication interface 410 may comprise, or be comprised by, human input/output interface 412. Communication interface 410 may be configured to communicate directly with a networked component or configured to utilize one or more networks, such as network 420 and/or network 424.

Network 420 may be a wired network (e.g., Ethernet), wireless (e.g., WiFi, Bluetooth, cellular, etc.) network, or combination thereof and enable device 402 to communicate with networked component(s) 422. In other embodiments, network 420 may be embodied, in whole or in part, as a telephony network (e.g., public switched telephone network (PSTN), private branch exchange (PBX), cellular telephony network, etc.).

Additionally or alternatively, one or more other networks may be utilized. For example, network 424 may represent a second network, which may facilitate communication with components utilized by device 402. For example, network 424 may be an internal network to a business entity or other organization, whereby components are trusted (or at least more so) than networked components 422, which may be connected to network 420 comprising a public network (e.g., Internet) that may not be as trusted.

Components attached to network 424 may include computer memory 426, data storage 428, input/output device(s) 430, and/or other components that may be accessible to processor 404. For example, computer memory 426 and/or data storage 428 may supplement or supplant computer memory 406 and/or data storage 408 entirely or for a particular task or purpose. As another example, computer memory 426 and/or data storage 428 may be an external data repository (e.g., server farm, array, “cloud,” etc.) and enable device 402, and/or other devices, to access data thereon. Similarly, input/output device(s) 430 may be accessed by processor 404 via human input/output interface 412 and/or via communication interface 410 either directly, via network 424, via network 420 alone (not shown), or via networks 424 and 420. Each of computer memory 406, data storage 408, computer memory 426, data storage 428 comprise a non-transitory data storage comprising a data storage device.

It should be appreciated that computer-readable data may be sent, received, stored, processed, and presented by a variety of components. It should also be appreciated that components illustrated may control other components, whether illustrated herein or otherwise. For example, one input/output device 430 may be a router, a switch, a port, or other communication component such that a particular output of processor 404 enables (or disables) input/output device 430, which may be associated with network 420 and/or network 424, to allow (or disallow) communications between two or more nodes on network 420 and/or network 424. One of ordinary skill in the art will appreciate that other communication equipment may be utilized, in addition or as an alternative, to those described herein without departing from the scope of the embodiments.

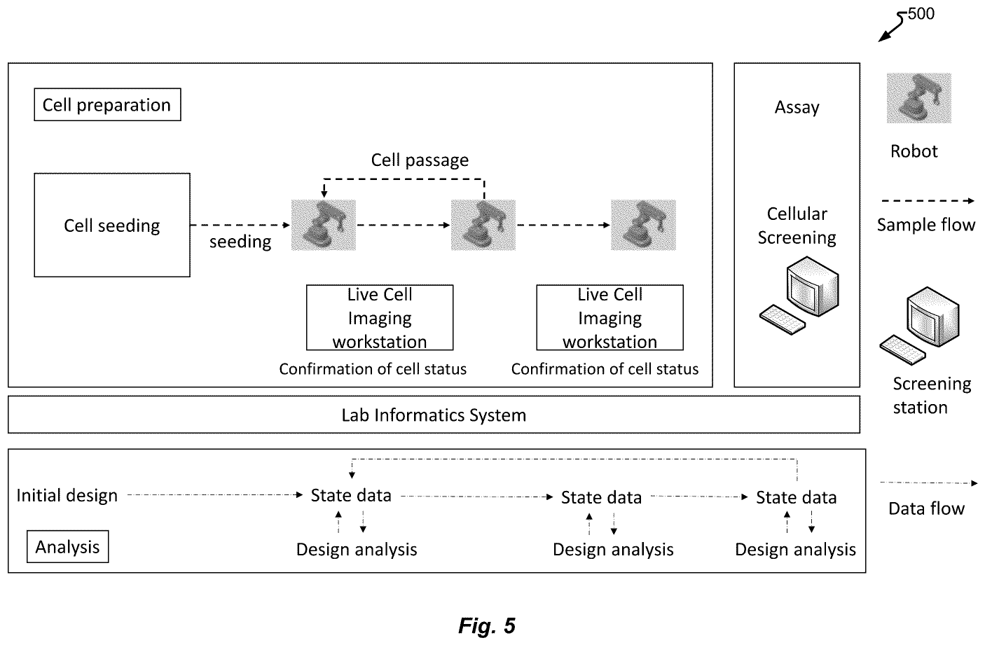

FIG. 5 FIG. 5 shows an example of automated cell preparation and drug screening 500 using a robot. In one embodiment, iPS cells are cultured and automatically differentiated for drug screening. In the case of cell culture, the template is initially set up with initial values for the medium used, medium conditions (glucose concentration, lactose concentration, etc.), culture temperature, CO2 concentration, culture time, plate mapping, and schedule for observation sensations, etc. At the same time, the number of viable cells to be reached, the number of cells to be differentiated, and the number of cells to be grown are also set up. At the same time, the number of viable cells reached, cell size, cell shape, etc. are set in advance. After the start of the experiment, the robot periodically sets the plate in the image analyzer, measures the number of viable cells, cell size, cell shape, etc., by image analysis, and analyzes the information by multivariate analysis, and then performs cell growth until the set upper limit value is reached. Cells that have reached the upper limit are cultured and observed repeatedly until the cell shape changes to that of the target cells. The cell proliferation and differentiation conditions are learned by AI and used to optimize the next and subsequent experiments. When the cells have sufficiently differentiated, the differentiated cells are used for drug screening experiments. For screening, the drug type, drug concentration, etc. are initially set in the template and added to each cell using an automated pipetting machine.

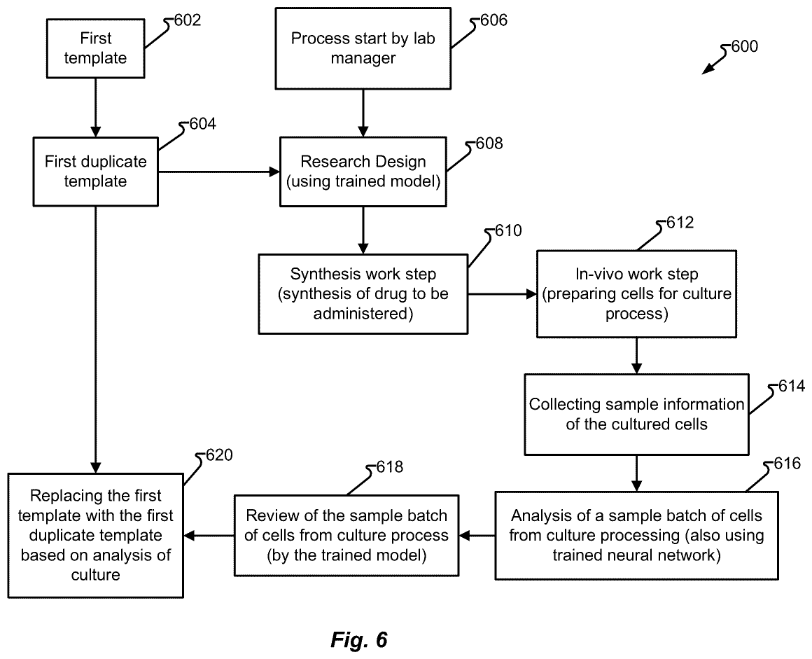

FIG. 6 depicts process 600 in accordance with embodiments of the present disclosure. In one embodiment, process 600 is embodied as machine-readable instructions maintained in a non-transitory memory that when read by a machine, such a processors of a server, cause the machine to execute the instructions and thereby execute process 600. The processor of the server may include, but is not limited to, at least one processor of server 116. In another embodiment, a human actor may initiate certain steps of process 600.

In step 606 a lab manager starts a resource process, such as process 300 (see FIG. 3). A first pharmaceutical workflow based on an existing template (e.g., a first template 602) is accessed.

Step 608 performs research design utilizing an AI (e.g., the trained neural network) and generates a schedule, measure, plate map of the end product intended to be created using the first template. Step 608 may be an embodiment of process 500 (see FIG. 5). The goal is to improve the first template, therefore first duplicated template 604 is created for modification along the pharmaceutical workflow. In the synthesis step, the drug is synthesized to be administered to the cell culture.

The synthesized step 610 formulation is administered to the cell culture. When the cell culture is set up and established, the data is collected both scheduled and unscheduled samples.

The cell cultures are collected based on the first template in step 614. Analysis step 616 allows study of the synthesized drug and its dosage applied to the in-vivo cell culture in step 612.

The result of analysis step is read by the trained neural network in step 618. In the case of drug screening, the study design team determines the culture conditions, reagent concentration conditions, media conditions, plate inoculation conditions, samples to be used, equipment to be used, and schedule based on their experience. The assigned team members perform the experiment according to the conditions and summarize the obtained results in a notebook. The analysis team summarizes the best results in a report and concludes the experiment. All of these flows depend on the experience and feeling of the person in charge, and it takes a great deal of effort and time to optimize all conditions and obtain optimal results.

In step 620, the result of experiments allows the research manager (AI model) to understand whether the experiment design leads to positive changes. In such a case, the changes are included into the first template.

In the foregoing description, for the purposes of illustration, methods were described in a particular order. It should be appreciated that in alternate embodiments, the methods may be performed in a different order than that described without departing from the scope of the embodiments. It should also be appreciated that the methods described above may be performed as algorithms executed by hardware components (e.g., circuitry) purpose-built to carry out one or more algorithms or portions thereof described herein. In another embodiment, the hardware component may comprise a general-purpose microprocessor (e.g., CPU, GPU) that is first converted to a special-purpose microprocessor. The special-purpose microprocessor then having had loaded therein encoded signals causing the, now special-purpose, microprocessor to maintain machine-readable instructions to enable the microprocessor to read and execute the machine-readable set of instructions derived from the algorithms and/or other instructions described herein. The machine-readable instructions utilized to execute the algorithm(s), or portions thereof, are not unlimited but utilize a finite set of instructions known to the microprocessor. The machine-readable instructions may be encoded in the microprocessor as signals or values in signal-producing components by, in one or more embodiments, voltages in memory circuits, configuration of switching circuits, and/or by selective use of particular logic gate circuits. Additionally or alternatively, the machine-readable instructions may be accessible to the microprocessor and encoded in a media or device as magnetic fields, voltage values, charge values, reflective/non-reflective portions, and/or physical indicia.

In another embodiment, the microprocessor further comprises one or more of a single microprocessor, a multi-core processor, a plurality of microprocessors, a distributed processing system (e.g., array(s), blade(s), server farm(s), “cloud”, multi-purpose processor array(s), cluster(s), etc.) and/or may be co-located with a microprocessor performing other processing operations. Any one or more microprocessors may be integrated into a single processing appliance (e.g., computer, server, blade, etc.) or located entirely, or in part, in a discrete component and connected via a communications link (e.g., bus, network, backplane, etc. or a plurality thereof).

Examples of general-purpose microprocessors may comprise, a central processing unit (CPU) with data values encoded in an instruction register (or other circuitry maintaining instructions) or data values comprising memory locations, which in turn comprise values utilized as instructions. The memory locations may further comprise a memory location that is external to the CPU. Such CPU-external components may be embodied as one or more of a field-programmable gate array (FPGA), read-only memory (ROM), programmable read-only memory (PROM), erasable programmable read-only memory (EPROM), random access memory (RAM), bus-accessible storage, network-accessible storage, etc.

These machine-executable instructions may be stored on one or more machine-readable mediums, such as CD-ROMs or other type of optical disks, floppy diskettes, ROMs, RAMs, EPROMS, EEPROMs, magnetic or optical cards, flash memory, or other types of machine-readable mediums suitable for storing electronic instructions. Alternatively, the methods may be performed by a combination of hardware and software.

In another embodiment, a microprocessor may be a system or collection of processing hardware components, such as a microprocessor on a client device and a microprocessor on a server, a collection of devices with their respective microprocessor, or a shared or remote processing service (e.g., “cloud” based microprocessor). A system of microprocessors may comprise task-specific allocation of processing tasks and/or shared or distributed processing tasks. In yet another embodiment, a microprocessor may execute software to provide the services to emulate a different microprocessor or microprocessors. As a result, a first microprocessor, comprised of a first set of hardware components, may virtually provide the services of a second microprocessor whereby the hardware associated with the first microprocessor may operate using an instruction set associated with the second microprocessor.

While machine-executable instructions may be stored and executed locally to a particular machine (e.g., personal computer, mobile computing device, laptop, etc.), it should be appreciated that the storage of data and/or instructions and/or the execution of at least a portion of the instructions may be provided via connectivity to a remote data storage and/or processing device or collection of devices, commonly known as “the cloud,” but may include a public, private, dedicated, shared and/or other service bureau, computing service, and/or “server farm.”

Examples of the microprocessors as described herein may include, but are not limited to, at least one of Qualcomm® Snapdragon® 800 and 801, Qualcomm® Snapdragon® 610 and 615 with 4G LTE Integration and 64-bit computing, Apple® A7 microprocessor with 64-bit architecture, Apple® M7 motion comicroprocessors, Samsung® Exynos® series, the Intel® Core™ family of microprocessors, the Intel® Xeon® family of microprocessors, the Intel® Atom™ family of microprocessors, the Intel Itanium® family of microprocessors, Intel® Core® i5-4670K and i-4770K 22 nm Haswell, Intel® Core® i5-3570K 22 nm Ivy Bridge, the AMD® FX™ family of microprocessors, AMD® FX-4300, FX-6300, and FX-8350 32 nm Vishera, AMD® Kaveri microprocessors, Texas Instruments® Jacinto C6000™ automotive infotainment microprocessors, Texas Instruments® OMAP™ automotive-grade mobile microprocessors, ARM® Cortex™-M microprocessors, ARM® Cortex-A and ARM926EJ-S™ microprocessors, other industry-equivalent microprocessors, and may perform computational functions using any known or future-developed standard, instruction set, libraries, and/or architecture.

Any of the steps, functions, and operations discussed herein can be performed continuously and automatically.

The exemplary systems and methods of this invention have been described in relation to communications systems and components and methods for monitoring, enhancing, and embellishing communications and messages. However, to avoid unnecessarily obscuring the present invention, the preceding description omits a number of known structures and devices. This omission is not to be construed as a limitation of the scope of the claimed invention. Specific details are set forth to provide an understanding of the present invention. It should, however, be appreciated that the present invention may be practiced in a variety of ways beyond the specific detail set forth herein.

Furthermore, while the exemplary embodiments illustrated herein show the various components of the system collocated, certain components of the system can be located remotely, at distant portions of a distributed network, such as a LAN and/or the Internet, or within a dedicated system. Thus, it should be appreciated, that the components or portions thereof (e.g., microprocessors, memory/storage, interfaces, etc.) of the system can be combined into one or more devices, such as a server, servers, computer, computing device, terminal, “cloud” or other distributed processing, or collocated on a particular node of a distributed network, such as an analog and/or digital telecommunications network, a packet-switched network, or a circuit-switched network. In another embodiment, the components may be physical or logically distributed across a plurality of components (e.g., a microprocessor may comprise a first microprocessor on one component and a second microprocessor on another component, each performing a portion of a shared task and/or an allocated task). It will be appreciated from the preceding description, and for reasons of computational efficiency, that the components of the system can be arranged at any location within a distributed network of components without affecting the operation of the system. For example, the various components can be located in a switch such as a PBX and media server, gateway, in one or more communications devices, at one or more users' premises, or some combination thereof. Similarly, one or more functional portions of the system could be distributed between a telecommunications device(s) and an associated computing device.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

Also, while the flowcharts have been discussed and illustrated in relation to a particular sequence of events, it should be appreciated that changes, additions, and omissions to this sequence can occur without materially affecting the operation of the invention.

A number of variations and modifications of the invention can be used. It would be possible to provide for some features of the invention without providing others.

In yet another embodiment, the systems and methods of this invention can be implemented in conjunction with a special purpose computer, a programmed microprocessor or microcontroller and peripheral integrated circuit element(s), an ASIC or other integrated circuit, a digital signal microprocessor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this invention. Exemplary hardware that can be used for the present invention includes computers, handheld devices, telephones (e.g., cellular, Internet enabled, digital, analog, hybrids, and others), and other hardware known in the art. Some of these devices include microprocessors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein as provided by one or more processing components.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this invention is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this invention can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Embodiments herein comprising software are executed, or stored for subsequent execution, by one or more microprocessors and are executed as executable code. The executable code being selected to execute instructions that comprise the particular embodiment. The instructions executed being a constrained set of instructions selected from the discrete set of native instructions understood by the microprocessor and, prior to execution, committed to microprocessor-accessible memory. In another embodiment, human-readable “source code” software, prior to execution by the one or more microprocessors, is first converted to system software to comprise a platform (e.g., computer, microprocessor, database, etc.) specific set of instructions selected from the platform's native instruction set.

Although the present invention describes components and functions implemented in the embodiments with reference to particular standards and protocols, the invention is not limited to such standards and protocols. Other similar standards and protocols not mentioned herein are in existence and are considered to be included in the present invention. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present invention.

The present invention, in various embodiments, configurations, and aspects, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments, configurations, or aspects for the purpose of streamlining the disclosure. The features of the embodiments, configurations, or aspects of the invention may be combined in alternate embodiments, configurations, or aspects other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the invention.

Moreover, though the description of the invention has included description of one or more embodiments, configurations, or aspects and certain variations and modifications, other variations, combinations, and modifications are within the scope of the invention, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

What is claimed is:

1. A method of optimizing a workflow relating to a work process, the method comprising:
identifying a plurality of work steps associated with the workflow;
creating a first template, wherein the first template comprises a first subset of the plurality of work steps;
duplicating the first template to create a first duplicated template;
modifying at least one of the first subset of the plurality of work steps automatically in the first duplicated template in accordance with a proposed modification workflow while maintaining a second subset of the plurality of work steps that remained unchanged;
automatically routing the proposed modification workflow through to the first duplicated template while allowing the workflow to be automatically routed to the first template;
validating the proposed modification workflow using the at least one of the first subset of the plurality of work steps and the second subset of the plurality of work steps that remained unchanged; and
replacing the first template with the first duplicated template when a result of validating the proposed modification workflow exceeds a predetermined threshold.

2. The method of claim 1, further comprising, forming a subset of a pharmaceutical screening workflow, the pharmaceutical screening workflow comprises:
a research work step of creating a proposed design schedule and a proposed formulation based on the first duplicated template; and
an analysis work step of validating the proposed design schedule and the proposed formulation based on an automatically generated analysis template that is generated from the first duplicated template.

3. The method of claim 2, wherein the pharmaceutical screening workflow further comprising: an in-vivo work step of preparing cells for culture process.

4. The method of claim 3, wherein the pharmaceutical screening workflow further comprising: a synthesis work step of preparation and synthesis of drug administration based on an automatically generated synthesis template generated from the first duplicated template.

5. The method of claim 4, wherein the in-vivo work step of preparing cells further comprises a first sub-step of receiving a synthesized drug sample from the synthesis work step, and a second sub-step of preparing dosage and a cell culture based on an information of the automatically generated synthesis template.

6. The method of claim 5, wherein the in-vivo work step of preparing cells further comprises a third sub-step of collecting sample information of the cell culture into a modified first duplicated template.

7. The method of claim 6, wherein validating the proposed modification workflow comprises fourth sub-step of analysis of a sample batch of cells from the cell culture.

8. The method of claim 7, wherein the proposed design schedule and the proposed formulation based on the first duplicated template are generated using a trained neural network.

9. The method of claim 8, wherein the trained neural network is used to perform a fifth sub-step of review of the sample batch of cells from the cell culture and in accordance with the proposed design schedule and the proposed formulation based on the first duplicated template.

10. A method of executing a workflow, comprising:
accessing a first workflow comprising a number of steps wherein each step of the number of steps comprises an assigned resource and a time for performance thereof;
creating a second workflow comprising a modification to the first workflow comprising at least a modification to the number of steps comprising adding a new step, a deleted step of the number of steps, or a modification to a step of the number of steps and wherein the modification to the first workflow reduces a workflow resource;
executing an executable workflow comprising the second workflow upon determining that, for the second workflow, each of the number of steps comprises the assigned resource that is indicated by a data record in a database as being available during the time; and
executing the executable workflow comprising the first workflow upon determining that, for the second workflow, each of the number of steps comprises the assigned resource that is indicated by the data record in the database as not available during the time.

11. The method of claim 10, wherein the creating the second workflow comprises providing the number of steps to a neural network trained to minimize utilization of a workflow resources for the first workflow when provided with the number of steps and, in response, returning the modification.

12. The method of claim 11, wherein the neural network is trained to determine a reduced resource utilization for a target workflow when provided with steps of the workflow, comprising:
accessing a set of past workflows, each comprising a plurality of past steps, and each past step having a corresponding past resource;
applying one or more transformations to each of the set of past workflows, including one or more of adding a step, removing a step, altering a past resource to utilize an equivalent resource able to perform the past step, utilizing more of a different resource to perform a step, utilizing a different input to perform a step, combining two or more past steps utilized by different resources into a single step utilized by a common resource, or utilizing less of the different resource to create a modified set of past steps;
creating a first training set comprising the set of past steps, the modified set of past steps, and a set of steps able to perform the target workflow that does not reduce resource utilization for the execution thereof;
training the neural network in a first training stage using the first training set;
creating a second training set for a second state of training comprising the first training set and the set of steps able to perform the target workflow that does not reduce resource utilization that was incorrectly identified as reducing resource utilization in the first training stage; and
training the neural network in the second state using the second training set.

13. The method of claim 10, wherein workflow resources of the workflow comprise at least one of a machine, a consumable input, time, a waste product, power, or variation of an output product.

14. The method of claim 10, wherein executing the executable workflow comprises allocating the assigned resource at a respective corresponding time.

15. The method of claim 10, wherein executing the executable workflow comprises executing at least one assigned resource at the respective corresponding time wherein the assigned resource comprises a machine.

16. The method of claim 10, wherein the time for one of the number of steps comprises a relative time determined by the completion of a prerequisite step of the number of steps.

17. The method of claim 10, further comprising saving the executable workflow as a template.

18. A system, comprising:
at least one processor coupled to computer memory comprising computer-executable instructions; and
wherein the at least one processor performs:
accessing a first workflow comprising a number of steps wherein each step of the number of steps comprises an assigned resource and a time for performance thereof;
creating a second workflow comprising a modification to the first workflow comprising at least a modification to the number of steps comprising adding a new step, a deleted step of the number of steps, or a modification to a step of the number of steps and wherein the modification to the first workflow reduces a workflow resource;
executing an executable workflow comprising the second workflow upon determining that, for the second workflow, each of the number of steps comprises the assigned resource that is indicated by a data record in a database as being available during the time; and
executing the executable workflow comprising the first workflow upon determining that, for the second workflow, each of the number of steps comprises the assigned resource that is indicated by the data record in the database as being not available during the time.

19. The system of claim 18, wherein the workflow resource comprises at least one of a machine, a consumable input, time, a waste product, power, or variation of an output product.

20. The system of claim 18, wherein the at least one processor performs executing the executable workflow further comprising allocating the assigned resource at the respective corresponding time and wherein the at least one processor performs creating the second workflow further comprising providing the number of steps to a neural network trained to minimize utilization of a workflow resources for the first workflow when provided with the number of steps and, in response, returning the modification.
